(19) United States
(12) Patent Application Publication
(10) Pub. No.: US 2020/0317533 A1
(43) Pub. Date: Oct. 8, 2020

(54) METHOD AND SYSTEM COMBINATION FOR THE PREPARATION OF SYNTHESIS PRODUCTS CONTAINING NITROGEN

(71) Applicant: LINDE AKTIENGESELLSCHAFT, Munchen (DE)

(72) Inventors: HEINZ ZIMMERMANN, Munchen (DE); helmut FRITZ, Munchen (DE); Ernst HAIDEGGER, Riemerling (DE); Rainer KEMPER, Munchen (DE)

(21) Appl. No.: 16/303,806

(22) PCT Filed: May 24, 2017
(86) PCT No.: PCT/EP2017/062672

§ 371 Date: Nov. 21, 2018

(51) Int. Cl. C01C 1/04; C01B 3/36; C07C 273/04; C07D 301/02; F25J 3/04

(52) U.S. Cl. CPC C01C 1/0405; C01B 3/36; C07C 273/04; C07D 301/02; F25J 3/04527; C01B 2203/0838; C01B 2203/0255; C01B 2203/1241; C01B 2203/068; C01B 2203/0883; F25J 3/04587

(57) ABSTRACT

The invention relates to a process (100), in which, with the inclusion of an air-separation method (10), an oxygen-rich substance flow (b) is formed, which, with a methane-rich substance flow (e), is subjected to a method for oxidative methane coupling. From the product flow (e) of the method for oxidative coupling of methane (20), one or more substance flows (f, i) are formed, which are subjected to one or more synthesis methods (40, 50) for the production of one or more nitrogen-containing synthesis products.

The invention relates to a process for the manufacture of nitrogen-containing synthesis products according to the preamble of claim 1.

### PRIOR ART

Methane, for example from natural gas, is currently used predominantly for burning. An alternative substance use is, however, of great interest from a commercial perspective. For example, methods for the manufacture of higher hydrocarbons from methane through oxidative coupling of methane (English: Oxidative Coupling of Methane, OCM) are currently being intensively developed. The oxidative coupling of methane is the direct conversion of methane in an oxidative, heterogeneously catalysed method to form higher hydrocarbons. Corresponding methods are used especially for the manufacture of ethylene.

For further details of the oxidative coupling of methane, reference is made to the relevant specialist literature, for example, Zavyalova, et al.: Statistical Analysis of Past Catalytic Data on Oxidative Methane Coupling for New Insights into the Composition of High-Performance Catalysts, ChemCatChem 3, 2011, 1935-1947. Methods for oxidative coupling of methane are e.g. also known from WO 2015/105911 A1 and WO 93/13039 A1.

In the oxidative coupling of methane, a methane-rich substance flow, for example, natural gas or a substance flow formed from natural gas, is supplied, together with an oxygen-rich substance flow, to a reactor. A product flow is formed which contains, alongside reaction products of the oxidative coupling of methane, especially ethylene, optionally propylene, hydrogen, carbon dioxide, unconverted methane and unconverted oxygen. If, for example, nitrogen-containing natural gas is used, the product flow will also contain nitrogen.

The oxygen-rich substance flow used for the oxidative coupling of methane is typically supplied through an air-separation method. The manufacture of air products by means of corresponding air-separation methods has been known for a considerable time and is described, for example, in H.-W. Häring (Ed.), Industrial Gases Processing, Wiley-VCH, 2006, especially subsection 2.2.5, “Cryogenic Rectification”. The present invention accordingly relates especially to such air-separation methods which are used for the production of gaseous, oxygen-rich substance flows.

In principle, there is a need to improve the exploitation of products from oxidative coupling of methane and to increase the overall yield from corresponding processes.

### DISCLOSURE OF THE INVENTION

This object is achieved by a process for the manufacture of nitrogen-containing synthesis products with the features of claim 1. In each case, further embodiments form the subject matter of the dependent claims and of the subsequent description.

In the conventional usage here, liquid and gaseous substance flows can be rich or poor in one or more components, wherein the term “rich” can stand for a content of at least 50%, 75%, 90%, 95%, 99%, 99.5%, 99.9% or 99.99%, and the term “poor” can stand for a maximum content of 50%, 25%, 10%, 5%, 1%, 0.1% or 0.01% on a molar, weight or volume basis.

If a substance flow is formed, in the conventional usage here, “with the inclusion” of a given method, for example, of an air-separation method or a method for oxidative coupling of methane, this explicitly does not exclude the participation of other methods, especially separation methods, in the formation of the substance flow. Similarly, the formulation does not exclude the formation of additional substance flows of the same or different composition through corresponding methods in each case.

### Advantages of the Invention

Against the background explained above, the present invention proposes a process in which, with the inclusion of an air-separation method, an oxygen-rich substance flow is formed, which is subjected, with a methane-rich substance flow, to a method for oxidative coupling of methane. To this extent, the process according to the invention does not differ from the processes of the prior art explained in the introduction. However, the invention now additionally proposes the formation, within such a process, from a product flow of the method for oxidative coupling of methane, of a hydrogen-rich substance flow and/or of a carbon-dioxide-rich substance flow, which is or are subjected to one or more synthesis methods for the production of one or more nitrogen-containing synthesis products.

As has been shown within the scope of the present invention, the coupling proposed according to the invention of the oxidative coupling of methane with corresponding synthesis methods is particularly suitable for increasing the overall efficiency of corresponding processes.

In this context, the process according to the invention can utilise, in the synthesis method or methods for the production of the nitrogen-containing synthesis products, a nitrogen-rich substance flow formed with the inclusion of the air-separation method and/or nitrogen which is contained in the product flow of the method for oxidative coupling of methane. If a nitrogen-rich substance flow formed with the inclusion of the air-separation method is subjected to the synthesis method, or to one of the synthesis methods for the production of the nitrogen-containing synthesis product or products, arbitrary quantities of nitrogen can be provided, so that the process is completely independent of nitrogen that might be contained only in small proportions or not at all in the waste flow of the method for oxidative coupling of methane. A corresponding process variant is therefore suitable especially for cases in which a product flow of the method for oxidative coupling of methane comprises no nitrogen content or an insufficient nitrogen content, or for the compensation of fluctuations in its nitrogen content.

In fact, temperatures and pressures such as are used in the synthesis methods for the production of nitrogen-containing synthesis products as explained in the following, for example, in ammonia-synthesis methods, are, in part, significantly higher than those used in the oxidative coupling of methane. However, the process according to the invention provides special advantages if the nitrogen which is contained in the product flow of the method for oxidative coupling of methane is used in a corresponding synthesis method for the production of a nitrogen-containing synthesis product. At least, no compression starting from atmospheric pressure and/or no temperature increase starting from ambient temperature or optionally below is necessary in this case, as might be required with the use of nitrogen from the air-separation method. The energy to be expended is therefore significantly reduced.

Synthesis methods for the production of nitrogen-containing synthesis products are known in principle. Within the scope of the present invention, especially ammonia synthesis and urea synthesis are of interest in this context. For details of both methods, reference is made to the specialist literature, for example, the article “Ethylene” in Ullmann's Encyclopedia of Industrial Chemistry, Online Publication 15th December 2006, doi:10.1002/14356007.a02_143.pub2, and the article “Urea” in Ullmann's Encyclopedia of Industrial Chemistry, Online Publication 10.1002/14356007.a27_333.pub2. Both in ammonia synthesis and also in urea synthesis, the process proposed within the scope of the present invention achieves special advantages.

If an ammonia-synthesis method is implemented within the scope of one embodiment of the invention, hydrogen required for this can originate from the product flow of the method for oxidative coupling of methane, as explained in the following, and/or from another method or respectively another source. For example, methane or natural gas which is also provided for the method for oxidative coupling of methane can also be subjected in parallel with this to a hydrogen synthesis method of known type, for example, a steam reforming. Hydrogen formed in this manner can be used alone or together with hydrogen which is contained in the product flow of the method for oxidative coupling of methane. In this manner, for example, an insufficient or fluctuating hydrogen content in the product flow of the method for oxidative coupling can be compensated. The preferred source for hydrogen is also determined by its accessibility. For example, if a recovery from the product flow of the method for oxidative coupling of methane proves too effort intensive, it is also possible to draw exclusively on hydrogen which is obtained by means of a separate hydrogen-synthesis method of the type explained.

Accordingly, a process in which the synthesis method or one of the synthesis methods is an ammonia-synthesis method, to which the hydrogen-rich substance flow which is formed in this variant of the method is subjected, is especially advantageous. The present invention accordingly allows an advantageous use of the hydrogen formed in the oxidative coupling of methane.

In particular, this process variant achieves special advantages in cases in which the product flow of the method for oxidative coupling of methane contains nitrogen, because this nitrogen need not then be separated from the hydrogen. A corresponding product flow can contain nitrogen for different reasons, wherein the process according to the invention is suitable in all such cases.

Accordingly, the present process proves advantageous especially in cases in which the methane-rich substance flow which is subjected to the method for oxidative coupling of methane, does not comprise only small quantities of nitrogen. Since this nitrogen is typically hardly converted or not converted at all in the method for oxidative coupling of methane, it is transferred into the product flow and must conventionally be separated in an effort intensive manner. With a boiling point of −196° C. (nitrogen) and −252° C. (hydrogen), nitrogen and hydrogen represent the components with the lowest boiling points in corresponding product flows. The other compounds contained in significant quantities in corresponding product flows boil at significantly higher temperatures. A separation of water and nitrogen would accordingly require, for example, an effort-intensive, low-temperature separation or a membrane process, which is disadvantageous for commercial reasons and/or would require effort-intensive additional separation equipment. The same also applies for a depletion of nitrogen in natural gas, which would have to be implemented in upstream method steps.

However, if a hydrogen-rich substance flow formed from a corresponding product flow is subjected to an ammonia synthesis method, any nitrogen contained is not problematic here. In this context, if the quantity of nitrogen contained in the product flow of the method for oxidative coupling of methane is not sufficient for the stoichiometric conversion with hydrogen, within the scope of the present invention, a further nitrogen-rich substance flow can be used, which can be formed, as explained previously, with the inclusion of the air-separation method.

If nitrogen-containing, methane-rich substance flows are used as feedstocks for the oxidative coupling of methane, these can comprise, for example, a nitrogen content of up to 20 mole percent, especially from 1 to 5 or 5 to 10 mole percent, wherein nitrogen contained in the methane-rich substance flow is transferred partially or completely into the hydrogen-rich substance flow and subjected to the ammonia-synthesis method within the latter. As explained previously, in this process variant, the present invention dispenses with a nitrogen depletion of the natural gas and/or a separation of hydrogen and nitrogen in a corresponding product flow.

However, in the embodiment explained, the present invention also allows the use of a more energy-efficient air-separation method, because pure oxygen in the form of the oxygen-rich substance flow need not necessarily be supplied to the method for oxidative coupling of methane. A less clear-cut separation of nitrogen from oxygen can also take place. As explained, in corresponding methods for oxidative coupling of methane, nitrogen is hardly converted or not converted at all, so that the latter is transferred into a corresponding product flow. The nitrogen contained in the product flow can then be used in an ammonia-synthesis method as explained. The present invention therefore also allows the use of oxygen-rich substance flows with a content of, for example, 1 to 20 mole percent, especially of 1 to 10 mole percent nitrogen. For example, the invention allows the use of air-separation plants with mixing columns. Corresponding plants and methods have been disclosed many times elsewhere, for example in EP 1 139 046 B1. For details on the optimisation of air-separation plants, reference is made to the relevant specialist literature, for example, Section 3.8 in Kerry, F. G., Industrial Gas Handbook: Gas Separation and Purification, Boca Raton: CRC Press, 2006.

Furthermore, in a product flow of a method for oxidative coupling of methane, carbon dioxide is typically present in considerable quantity. Corresponding carbon dioxide is typically removed from corresponding substance flows upstream of a separation method, in order to prevent freezing out and accordingly displacement of plant components at the separating temperatures and pressures used. For the separation of carbon dioxide, a per se known carbon-dioxide scrubbing can be used.

The carbon dioxide obtained in this context is particularly suitable for use in a urea-synthesis method. In this context, it is particularly advantageous if an ammonia-synthesis method is initially implemented, as explained previously. Ammonia, which is formed in a corresponding ammonia-synthesis method, can then be converted with the carbon dioxide to form urea, as known in principle. In the process variant proposed, the process allows, for example, a further integration of air separation and oxidative coupling of methane. Within the scope of the present invention, it therefore proves advantageous if the synthesis method or one of the synthesis methods is a urea-synthesis method, to which the carbon-dioxide-rich substance flow which is formed in this variant of the method is subjected.

From a conventional perspective, the carbon dioxide is an undesirable by-product. As explained, for example, by Zavyalova et al. (see above), in the oxidative coupling of methane, a non-selective oxidation of the methane and of the hydrocarbons formed occurs to give carbon monoxide and carbon dioxide. Conventionally, therefore, appropriate catalysts for the oxidative coupling of methane should catalyse not only the formation of methyl radicals, which then react to form ethane and ethylene, but should also suppress the non-selective oxidation of methane and of the hydrocarbons formed. If a method according to the embodiment just explained is used, carbon dioxide can be converted in its entirety to form products, so that this aspect has a relatively low significance and greater freedoms are achieved in the optimisation of a corresponding catalyst.

However, the present invention allows an even greater integration of the named process components and respectively methods. Accordingly, it is particularly advantageous if, from the product flow of the method for oxidative coupling of methane, one or more olefin-rich substance flows and, with the inclusion of the air-separation method, one or more further oxygen-rich substance flows are formed. The olefin-rich substance flow or flows and the further oxygen-rich substance flow or flows can be subjected together with one another to an epoxidation method. Corresponding epoxidation methods can be provided separately for an olefin-rich substance flow and or for several in combination. In particular, only one olefin-rich substance flow may be epoxidised. One or more corresponding olefin-rich substance flows are rich in ethylene and/or propylene. With a corresponding epoxidation, propylene oxide is formed from propylene, and ethylene oxide is formed from ethylene, that is, compounds which are particularly suitable as starting components for further reactions. In particular, it can be provided to synthesise ethylene glycol and/or propylene glycol from the ethylene oxide and/or propylene oxide formed in the epoxidation method.

The present invention also allows the recycling of substance flows, for example, by forming at least one further substance flow from the product flow of the method for oxidative coupling of methane, which is again subjected to the method for oxidative coupling of methane. Advantageously in this context, the at least one further substance flow which can, in particular, contain methane, is poor in or free from nitrogen, however, it can contain nitrogen, if, within the framework of the method according to the invention, nitrogen is drawn continuously from a corresponding circulation, that is, for example, supplied to the ammonia-synthesis method.

The invention also allows an advantageous thermal integration, namely, an exploitation of the waste heat of the method for oxidative coupling of methane for the pre-heating or heating of substance flows or reactors which are used for the synthesis of the nitrogen-containing product or products.

The present invention can envisage the use of a combined plant, which comprises an air-separation plant and at least one reactor equipped for the implementation of a method for the oxidative coupling of methane. The combined plant comprises means, which are equipped, with the inclusion of an air-separation method implemented in the air-separation plant, to form an oxygen-rich substance flow and to subject the latter with a methane-rich substance flow to a method for oxidative coupling of methane in the at least one reactor. Advantageously, means are provided which are equipped, from a product flow of the method for oxidative coupling of methane, to form one or more substance flows and to subject this substance flow or flows, in one or more further reactors to one or more synthesis methods for the production of one or more nitrogen-containing synthesis products.

A corresponding combined plant is advantageously equipped for the implementation of a method as was explained previously in detail and, for this purpose, comprises correspondingly equipped means. Regarding features and advantages of the corresponding plant complex, reference is therefore made explicitly to the above explanations.

In the following, the invention is explained in greater detail with reference to the attached drawing, which shows preferred embodiments of the invention

### BRIEF DESCRIPTION OF THE DRAWING

FIG. 1 shows a process for manufacturing reaction products according to a particularly preferred embodiment of the invention.

### DETAILED DESCRIPTION OF THE DRAWING

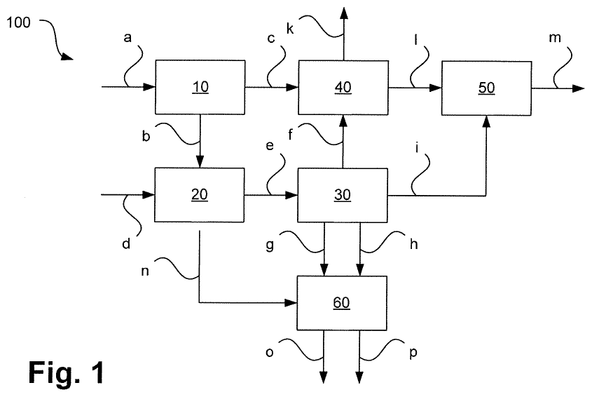

In FIG. 1, a process according to a particularly preferred embodiment of the invention is illustrated in the form of a schematic process-flow diagram and designated as a whole as 100.

The process 100 comprises an air-separation method 10 and a method for oxidative coupling of methane 20. Feedstock air in the form of a substance flow a is supplied to the air-separation method 10. Air-separation methods 10 suitable for use within the scope of the process 100 have been described extensively elsewhere.

With the use of a corresponding air-separation method 10 in the illustrated example, an oxygen-rich substance flow b and a nitrogen-rich substance flow c are provided. However, with the use of the air-separation method 10, arbitrary further substance flows, which can comprise air-separation products, can also be provided, for example, further oxygen-rich and/or nitrogen-rich substance flows and/or substance flows which are rich in one or more noble gases, as is known in principle.

In the illustrated example, the oxygen-rich substance flow b and a methane-rich substance flow d, which can be, for example, conditioned or unconditioned natural gas, are supplied to the method for oxidative coupling of methane 20. In the method for oxidative coupling of methane 20, a product flow e is produced, which can contain, inter alia, unconverted methane of the substance flow d, unconverted oxygen of the substance flow b, inert gases such as nitrogen optionally contained in the substance flow d, and reaction products of the oxidative coupling of methane, such as hydrogen, carbon dioxide, ethylene or propylene.

The product flow e is subjected to a separation method 30, which can comprise non-cryogenic and cryogenic separation steps. In particular, the separation method 30 can also comprise a gas scrubbing. With the use of the separation method 30, in particular, a hydrogen-rich substance flow f, an ethylene-rich substance flow g, a propylene-rich substance flow h and a carbon-dioxide-rich substance flow i can be provided. The hydrogen-rich substance flow f, the propylene-rich substance flow g and the ethylene-rich substance flow h are typically produced in one or more cryogenic separation steps of the separation method 30. The carbon-dioxide-rich substance flow i is typically separated in advance. In the separation method 30 or respectively in corresponding separation steps, further substance flows can also be provided, which have, however, not been shown in FIG. 1 for the sake of visual clarity.

The core of the process 100 in the particularly preferred embodiment shown in FIG. 1 is the implementation of an ammonia-synthesis method 40, to which the nitrogen-rich substance flow c, which is prepared with the use of the air-separation method 10, and the hydrogen-rich substance flow f, which is prepared with the use of the method for oxidative coupling of methane and the downstream separation method 30, are supplied in the illustrated example. It should be emphasised that, with the use of the method for oxidative coupling of methane 20 or respectively of the downstream separation method 30, further hydrogen-rich flows can also be provided, which need not necessarily be supplied in their entirety to the ammonia-synthesis method 40. Similarly, the nitrogen supplied to the ammonia synthesis method 40 need not originate or need not originate exclusively from the nitrogen-rich substance flow c from the air-separation method 10. At least a part of the nitrogen can also be contained in the hydrogen-rich substance flow f, as explained above.

With the use of the ammonia-synthesis method 40 in the example shown, two ammonia-rich flows k and l are provided. By contrast with the particularly preferred embodiment of the process 100 according to the invention shown in FIG. 1, ammonia can also represent a single nitrogen-containing end-product of a corresponding process. In this case, further method steps for the conversion of ammonia, as are implemented in FIG. 1 and explained in the following, are dispensed with.

However, the particularly preferred embodiment of the process 100 shown in FIG. 1, comprises a urea synthesis method 50. In this context, the ammonia-rich flow l, which is provided with the use of the ammonia-synthesis method 40, and the carbon-dioxide-rich flow i, which is provided with the use of the method for oxidative coupling of methane 20 and the downstream separation method 30, are supplied to the urea-synthesis method 50. It goes without saying that the entire ammonia formed in the ammonia-synthesis step 40 and/or the entire carbon dioxide provided in the method for oxidative coupling of methane 20 and the downstream separation method 30 need not be supplied to the urea-synthesis method 50. In each case, only partial quantities of the named compounds can also be used; the remainder can be supplied from a corresponding process 100, for example, as a product or respectively by-product. A corresponding case is shown in FIG. 1 with the ammonia-rich substance flows k and l.

In the illustrated example, the ammonia-rich substance flow k is output from the process. With the use of the urea-synthesis method 50 in the particularly preferred embodiment of the invention shown in FIG. 1, a urea-rich substance flow m is provided and supplied as required to suitable conditioning steps.

The methods explained in the following are also not necessarily a component of a corresponding process 100. This means that the propylene-rich substance flow g and/or the ethylene-rich substance flow h can also be output from a corresponding process 100, in each case.

In the illustrated example, an epoxidation method 60 is shown, which can also be provided separately for the propylene-rich substance flow g and the ethylene-rich substance flow h or only for one of these substance flows. An oxygen-rich substance flow n, which can, in particular, be provided with the use of the air-separation method 10, is supplied to the epoxidation method 60. With the use of the epoxidation method 60, a propylene-oxide-rich substance flow o and/or an ethylene-oxide-rich substance flow p can be provided. Here also, the entire propylene and or ethylene provided in the method for oxidative coupling of methane 20 or respectively the downstream separation method 30 need not be subjected to the epoxidation method 60. In particular, partial flows of corresponding propylene or respectively ethylene can be output from the process 100 as products.

1. A process, in which, with the inclusion of an air-separation method, an oxygen-rich substance flow is formed, which is subjected, with a methane-rich substance flow, to a method for oxidative coupling of methane, characterised in that, from a product flow of the method for oxidative coupling of methane, a hydrogen-rich substance flow and/or a carbon-dioxide-rich substance flow are formed, and that the substance flow or flows formed from the product flow are subjected to one or more synthesis methods for the production of one or more nitrogen-containing synthesis products.

2. The process according to claim 1, in which, with the inclusion of the air-separation method, furthermore, a nitrogen-rich substance flow is formed, wherein the nitrogen-rich substance flow is subjected to the, or to one of the, synthesis methods.

3. The process according to claim 1, in which the hydrogen-rich substance flow is formed, wherein the, or one of the, synthesis methods is an ammonia-synthesis method, to which the hydrogen-rich substance flow is subjected.

4. The process according to claim 3, in which the methane-rich substance flow contains nitrogen, wherein the nitrogen contained in the methane-rich substance flow is partially or completely transferred into the hydrogen-rich substance flow and, within the latter, subjected to the ammonia-synthesis method.

5. The process according to claim 4, in which the methane-rich substance flow contains up to 20 mole percent nitrogen.

6. The process according to any one of claim 3, in which the oxygen-rich substance flow contains nitrogen, wherein the nitrogen contained in the oxygen-rich substance flow is partially or completely transferred into the hydrogen-rich substance flow and, within the latter, subjected to the ammonia-synthesis method.

7. The process according to claim 6, in which the oxygen-rich substance flow contains up to 20 mole percent nitrogen.

8. The process according to claim 1, in which carbon-dioxide-rich substance flow is formed, wherein the, or one of the, synthesis methods is a urea synthesis method, to which the carbon-dioxide-rich substance flow is subjected.

9. The process according to claim 1, in which, from the product flow, furthermore, one or more olefin-rich substance flows are formed, and, with the inclusion of an air separation method, one or more further oxygen-rich substance flows are formed, wherein the olefin-rich substance flow or flows and/or the further oxygen-rich substance flow or flows are subjected to an epoxidation method.

10. The process according to claim 1, in which, from the product flow, at least one further substance flow is formed, which, is again subjected to the method for oxidative coupling of methane.

11. The process according to claim 1, in which the waste heat of the method for oxidative methane coupling is used for the pre-heating or heating of one or more substance flows and/or of one or more reactors, which are used in the synthesis method for the production of the nitrogen-containing synthesis product or products.

12. The process according to claim 4, in which the methane-rich substance flow contains from 5 to 10 mole percent nitrogen.

13. The process according to claim 6, in which the oxygen-rich substance flow contains from 5 to 10 mole percent nitrogen.
